(19) United States
(12) Patent Application Publication
(10) Pub. No.: US 2026/0157778 A1
(43) Pub. Date: Jun. 11, 2026

(54) INTRAMEDULLARY NAIL WITH CONTINUOUS COMPRESSION

(71) Applicant: DePuy Synthes Products, Inc., Raynham, MA (US)

(72) Inventors: Ross Hamel, West Chester, PA (US); Rene Haag, Berwyn, PA (US)

(21) Appl. No.: 19/375,687

(22) Filed: Oct. 31, 2025

Related U.S. Application Data
(60) Provisional application No. 63/714,159, filed on Oct. 31, 2024.

(51) Int. Cl. A61B 17/72

(52) U.S. Cl. CPC A61B 17/7225

(57) ABSTRACT

An intramedullary nail can extend along a central axis. The intramedullary nail can include a first nail member, a second nail member, and a biasing element. The first nail member can be configured to be positioned in a medullary canal of a bone. The first nail member can define at least one first bone fixation hole configured to receive a respective at least one first bone fixation element so as to couple the first nail member to a first bone portion of the bone. The second nail member can be configured to be at least partially inserted into the first nail member. The second nail member can define at least one second bone fixation hole configured to receive a respective at least one second bone fixation element so as to couple the second nail member to a second bone portion of the bone that is spaced from the first bone portion. The biasing element can be configured to apply a biasing force to at least one of the first and second nail members that creates continuous compression between the first bone portion and the second bone portion. In some examples, the biasing force can decrease a distance from the at least one first bone fixation hole to the at least one second bone fixation hole along the central axis.

### CROSS-REFERENCE TO RELATED APPLICATIONS

This claims the benefit of U.S. Patent Application Ser. No. 63/714,159 filed Oct. 31, 2024, the disclosure of which is hereby incorporated by reference as if set forth in its entirety herein.

### TECHNICAL FIELD

The present disclosure relates generally to bone fixation devices, and more specifically to intramedullary nails to couple bone segments to each other.

### BACKGROUND

Conventional intramedullary nails are configured to be inserted into the medullary canal of a bone that has been fractured so as to define a proximal bone segment and a distal bone segment that is separated from the proximal bone segment by a bone gap. Conventional intramedullary nails are elongate along a substantially central longitudinal axis, and include bone anchor holes that are configured to receive bone anchors. The bone anchor holes can include proximal bone anchor holes that extend through the proximal end of the intramedullary nail and distal bone anchor holes that extend through the distal end of the intramedullary nail. Thus, the intramedullary nail can be inserted into the medullary canal of the fractured long bone such that the proximal bone anchor holes are aligned with the proximal bone segment and the distal bone anchor holes are aligned with the distal bone segment on opposite sides of the bone gap. The bone segments can be positioned by a surgeon and the bone screws can be driven into the bone segments and the corresponding bone anchor holes so as to fasten the intramedullary nail to the fractured long bone and stabilize the proximal and distal bone segments relative to each other, thereby promoting healing. The relative positions of the bone segments are fixed once the bone screws are set using such traditional designs. Therefore, an improved intramedullary nail that compresses the bone segments against each other during the healing process is desired.

### SUMMARY

In one example, an intramedullary nail can extend along a central axis. The intramedullary nail can include a first nail member configured to be positioned in a medullary canal of a bone. The first nail member can define at least one first bone fixation hole configured to receive a respective at least one first bone fixation element so as to couple the first nail member to a first bone portion. The intramedullary nail can further include a second nail member configured to be at least partially inserted into the first nail member. The second nail member defines at least one second bone fixation hole configured to receive a respective at least one second bone fixation element so as to couple the second nail member to a second bone portion that is spaced from the first bone portion. The intramedullary nail can further include a biasing element configured to apply a biasing force to at least one of the first and second nail members so as to apply compression between the first and second bone portions when the first nail member is coupled to the first bone portion and the second nail member is coupled to the second bone portion. In some examples, the biasing force can decrease a distance from the at least one first bone fixation hole to the at least one second bone fixation hole along the central axis.

### BRIEF DESCRIPTION OF THE DRAWINGS

The present application is further understood when read in conjunction with the appended drawings. For the purpose of illustrating the subject matter, there are shown in the drawings exemplary aspects of the subject matter; however, the presently disclosed subject matter is not limited to the specific methods, devices, and systems disclosed. In the drawings:

FIG. 1 is a top plan view of an intramedullary nail in accordance with one embodiment of the present disclosure;

FIG. 2 is a sectional view of a first nail member of the intramedullary nail of FIG. 1;

FIG. 3 is a sectional view of the intramedullary nail of FIG. 1 in an initial configuration;

FIG. 4 is a sectional view of the intramedullary nail of FIG. 1 in an intermediate configuration;

FIG. 5 is a sectional view of the intramedullary nail of FIG. 1 in a reduced configuration;

FIG. 6 is a flow chart illustrating a method of assembling the intramedullary nail of FIG. 1; and

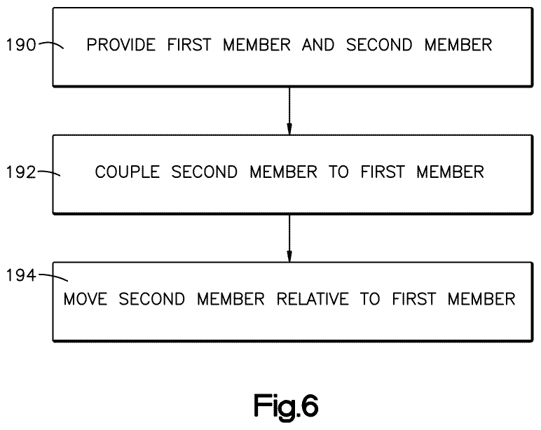

FIG. 7 is a side elevation view showing a pull tool configured to compress a biasing element of the intramedullary nail of FIG. 1.

Aspects of the disclosure will now be described in detail with reference to the drawings, wherein like reference numbers refer to like elements throughout, unless specified otherwise.

### DETAILED DESCRIPTION

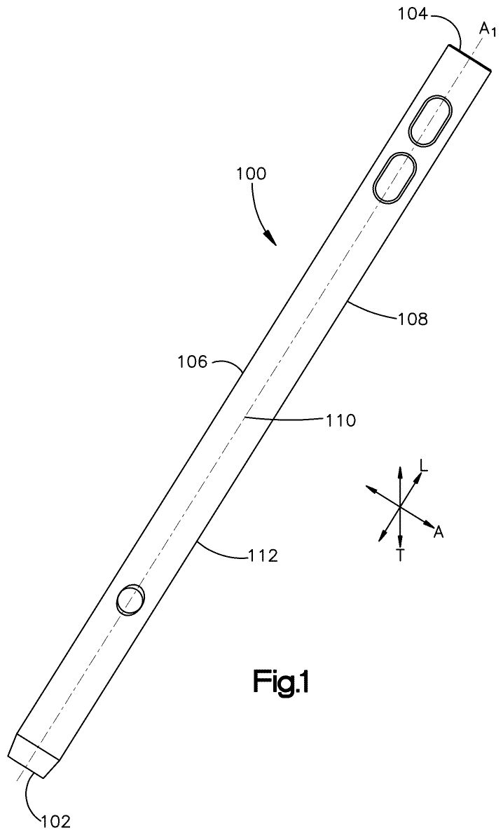

Referring to FIG. 1, a fixation device 100 is configured to couple first and second bone portions to each other. In one example, the fixation device 100 can apply a force to either or both of the bone portions that causes either or both of the bone portions to move toward the other of the bone portions. For instance, the fixation device 100 can move either of both of the bone into contact with the other bone portion. The fixation device 100 can provide a compression force that compresses the bone portions against each other. The fixation device 100 can move the bone portions into contact with each other such that the first and second bone portions exert a compressive force against each other. The first and second bone portions can thus exert a continuous compressive force against each other during the healing process.

In some embodiments, the fixation device 100 can be coupled to an outer surface of the bone portions. In other embodiments, the fixation device 100 can be positioned in a medullary canal. The fixation device 100 can be an intramedullary nail. The fixation device 100 can be positioned in the medullary canal and the bone portions can be coupled to the fixation device 100 to maintain the position of the bone portions under compression during the healing process.

In one example, the bone portions can be defined by single fractured bone. The fracture can be caused by trauma or osteotomy cut. In other examples, the bone portions can be defined by two different bones that are to be coupled together. The bone or bones can be defined by one or more long bones, such as for example a tibia, fibula, femur, metatarsals, and phalange, humerus, radius, ulna, or metacarpal.

Referring to FIG. 1, the fixation device 100 can define a distal end 102 and a proximal end 104 spaced from the proximal end along a longitudinal direction L. The distal end 102 is spaced from the proximal end 104 in a distal direction. Conversely, the proximal end 104 is spaced from the distal end 102 in a proximal direction that is opposite the distal direction. Each of the proximal and distal directions can be oriented along the longitudinal direction L. The fixation device 100 can include a first side 106 and a second side 108 spaced from the first side 106 along a lateral direction A. The fixation device 100 can include a first margin 110 and a second margin 112 spaced from the first margin 110 along a transverse direction T. The lateral direction A can be perpendicular to the longitudinal direction L. The transverse direction T can be perpendicular to each of the longitudinal direction L and the lateral direction A. The fixation device 100 can be elongate along a central axis A₁. The central axis A₁ can be oriented along the longitudinal direction L.

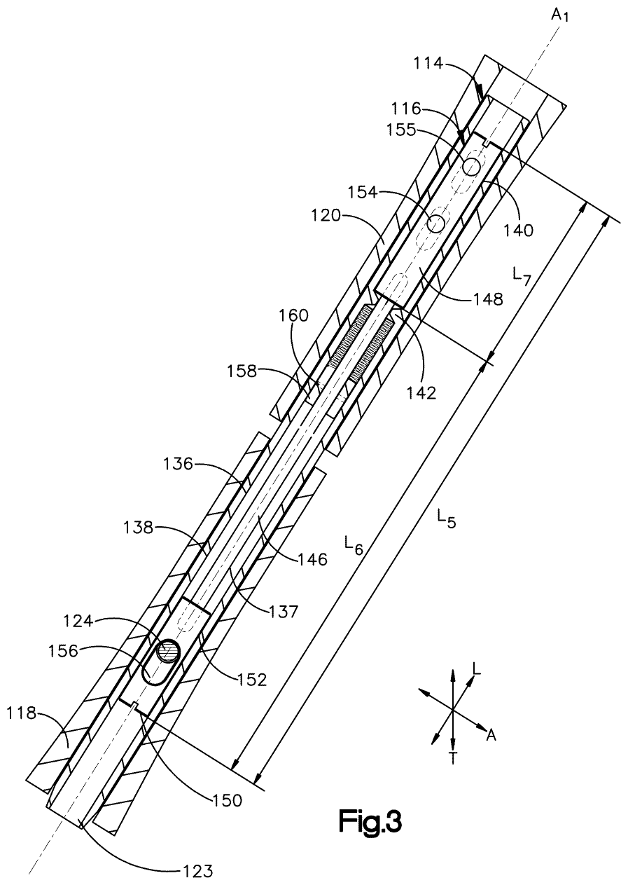

The fixation device 100 can include a first nail member 114 and a second nail member 116 (FIG. 3). The first nail member 114 can be coupled to a first bone portion 118. The second nail member 116 can be coupled to a second bone portion 120. The first nail member 114 and second nail member 116 can be movable relative to each other to move the bone portions. The first bone portion 118 can move relative to the second bone portion 120 as the first nail member 114 moves relative to the second nail member 116. The first nail member 114 and second nail member 116 can be movable relative to each other in the longitudinal direction L. At least one of the first nail member 114 and second nail member 116 can be movable relative to the other of the first nail member 114 and second nail member 116. At least one of the first nail member 114 and second nail member 116 can be movable relative to the other of the first nail member 114 and second nail member 116 in the longitudinal direction L. At least one of the first nail member 114 and second nail member 116 can be movable relative to the other of the first nail member 114 and second nail member 116 along the longitudinal axis A₁. Either or both of the first nail member 114 and the second nail member 116 can be a single unitary monolithic structure, or can be defined by two or more pieces.

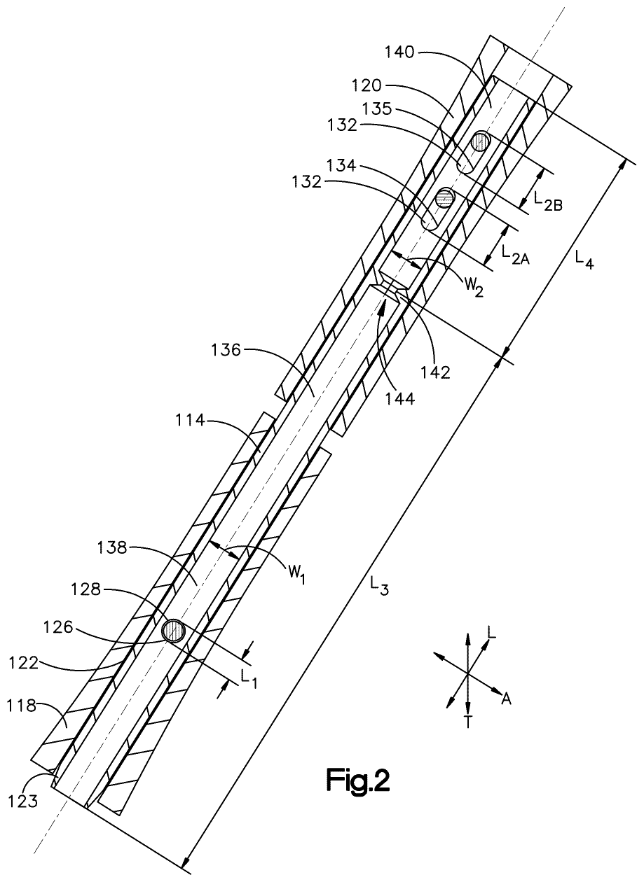

In some examples, the first and second nail members 114, 116 are positioned next to each other in a medullary canal of a long bone that defines the first and second bone portions 118, 120. In other examples, one of the first and second nail members 114, 116 is nested within the other of the first and second nail members 114, 116. Referring now to FIGS. 2-3, the first nail member 114 can include an outer wall 122. The outer wall 122 can define a channel configured to receive at least a portion of the second nail member 116. The outer wall 122 can be sized and shaped such that the first nail member 114 can be inserted into the medullary canal. The outer wall 122 can have a circular cross-sectional shape taken along a plane that is oriented transverse to the longitudinal direction L. Thus, the plane can extend in the transverse direction T and the lateral direction A. The outer wall 122 can define a cylindrical shape. The first nail member 114 can include an end portion 123 configured to facilitate insertion of the first nail member 114 into the medullary canal. The end portion 123 can be tapered inwardly in the longitudinal direction to facilitate insertion into the medullary canal. In this regard, the end portion 123 can define a leading end of the first nail member 114. The end portion 123 can be tapered inwardly in a direction from the proximal end 104 toward the distal end 102.

The first nail member 114 can be configured to be fixed to the first bone portion 118. As is described in more detail below, the first nail member 114 can be translatably fixed to the first bone portion 118, such that movement of the first nail member 114 along the longitudinal direction L correspondingly causes the first bone portion 118 to move along the longitudinal direction. The first nail member 114 can be configured to be fixed to the first bone portion 118 by a first bone fixation element 124 (FIG. 3). The first bone fixation element 124 can be a threaded fastener, rivet, nail, or adhesive. The first nail member 114 can include a first aperture 126 in the outer wall 122 configured to receive the first bone fixation element 124. The first aperture 126 can be a first bone fixation hole. The first aperture 126 can extend through the outer wall 122 from a first side of the outer wall to a second side of the outer wall 122 along an first aperture central axis. The first aperture central axis can be transverse to the central axis A₁. The first aperture central axis can be oriented along the transverse direction T, the lateral direction A, or any alternative suitable direction that includes each of the transverse direction T and the lateral direction A. The first aperture 126 can be aligned with a first bone hole 128 (FIG. 2). The first aperture 126 can have a length L₁ in the longitudinal direction L. The first aperture 126 can be cylindrical in some examples, whereby the length L₁ defines a diameter of the first aperture 126. In some examples, the first aperture 126 can be threaded.

The first bone fixation element 124 can fix the position of the first nail member 114 in the longitudinal direction L relative to the first bone portion 118. The first bone fixation element 124 can be positioned in the first aperture 126. The first bone fixation element 124 can extend through the first aperture 126. The first bone fixation element 124 can extend through the first aperture 126 from an outer surface of the first nail member 114 toward the central axis A₁. The first bone fixation element 124 can extend from an outer surface of the first nail member 114 to the central axis A₁. The first bone fixation element 124 can extend through the first nail member 114. The first bone fixation element 124 can extend from a first outer surface of the outer wall 122 to a second outer surface of the outer wall 122 opposite the first outer surface. The first bone fixation element 124 can extend through the first bone hole 128 into the first aperture 126. The first bone fixation element 124 can have a shaft that is threaded, and can threadedly mate with the first bone portion 118 in the first aperture 126. The first bone fixation element 124 can include a head that extends radially outward relative to the shaft, and engages a sidewall of the first aperture 126 thereby locking the first fastener 125 in the first aperture 126. The first aperture 126 and the first bone fixation element 124 can be of generally equal length L₁ in the longitudinal direction L. The length L₁ can define an outer diameter of the shaft of the first bone fixation element 124. The first aperture 126 can be sized and shaped to receive the first bone fixation element 124 such that the first nail member 114 is positionally fixed in the longitudinal direction L relative to the first bone portion 118. Although only one first aperture 126 is shown in the figures, any number (e.g., two, three, four) of first apertures 126 can be incorporated into the first nail member 114 that are configured to receive respective first bone fixation element 124 so as to fix the first nail member 114 to the first bone portion 118.

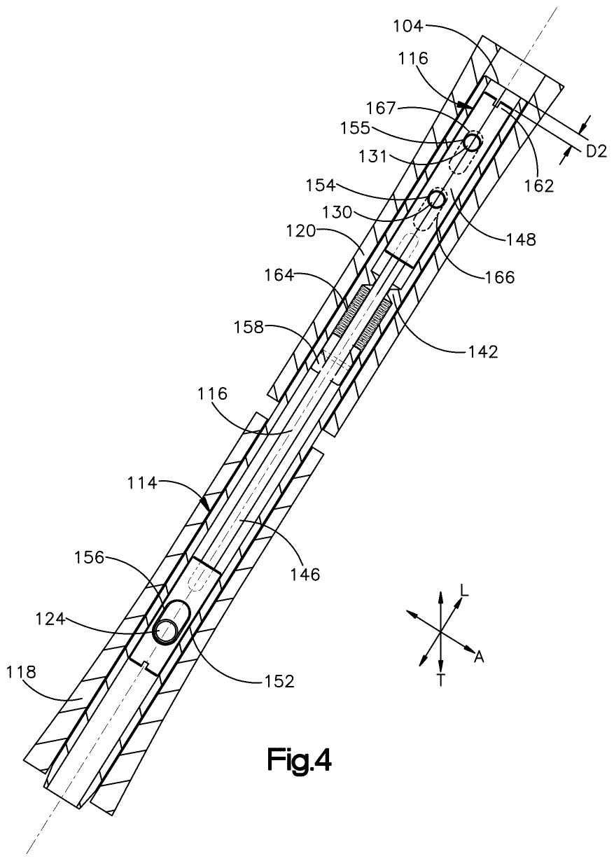

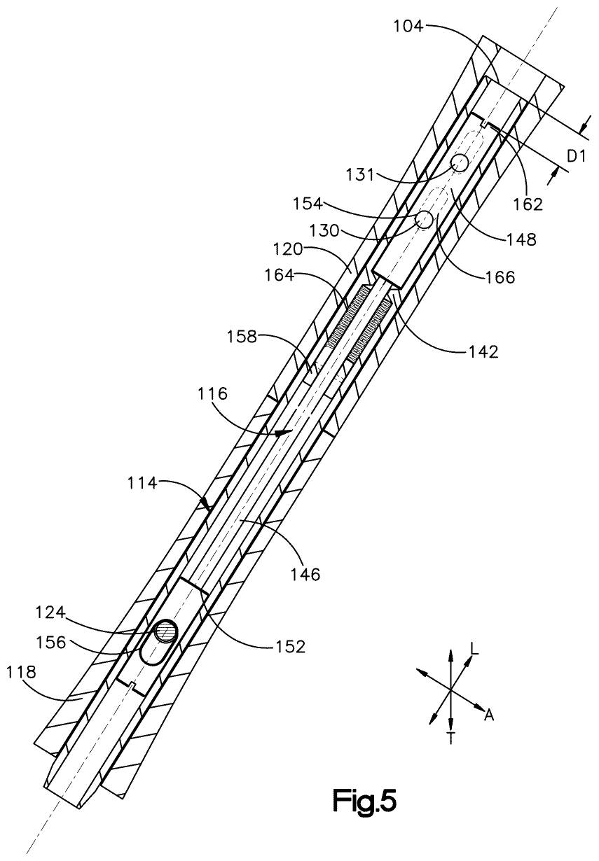

The first nail member 114 can be further configured to be coupled to the second bone portion 120, such that the first nail member 114 is translatable with respect to the second bone portion 120 along the longitudinal direction. In one example, the first nail member 114 can be configured to receive a second bone fixation element 130 (FIG. 5). The second bone fixation element 130 can be coupled to the second bone portion 120. For instance, the second bone fixation element 130 can be drilled through a hole in the second bone portion 120, or can form a hole through the second bone portion 120 as it is driven through the second bone portion 120. The second bone fixation element 130 can be translatably fixed to the second bone portion 120 with respect to relative movement along the longitudinal direction L The first nail member 114 can include a first slot 132 (FIG. 2) configured to receive the second bone fixation element 130. The first slot 132 can be elongate, for instance along the longitudinal direction L. A The first nail member 114 can include a first slot sidewall 134 that defines the first slot 132. The first slot 132 can extend through the outer wall 122 of the first nail member 114. The first slot 132 can be elongate in the longitudinal direction L such that the first nail member 114 is moveable in the longitudinal direction L relative to the second bone fixation element 130 when the second bone fixation element 130 extends through the first slot 132. The first slot 132 can have a length L_{2A} in the longitudinal direction. The length L_{2A} can be greater than the diameter of the second bone fixation element 130. In one example, the first and second bone fixation elements 124 and 130 can have the same diameter. In other examples, the first and second bone fixation elements 124 and 130 can have different diameters. The second bone fixation element 130 can be movable within the first slot 132 from a respective first position (FIG. 4) to a respective second position (FIG. 5). In one example, movement of the second bone fixation element 130 in the first slot 132 can be defined by a translation of the second bone fixation element 130 along the longitudinal direction L. Accordingly, the first nail member 114 can be movable relative to the second bone fixation element 130, and thus relative to the second bone portion 120, from a first position (FIG. 4) to a second position (FIG. 5). The second bone fixation element 130 can translate in the first slot 132 as the first nail member 114 moves relative to the second bone fixation element 130, and thus relative to the second bone portion 120. The second bone fixation element 130 can contact a distal portion of the first slot sidewall 134 in the first position. The second bone fixation element 130 can contact a proximal portion of the first slot sidewall 134 in the second position. Alternatively, the second bone fixation element 130 can be spaced from the proximal end of the first slot 132 in one or both of the first and second positions. The second position of the second bone fixation element 130 relative to the first slot 132 can be offset from the first position of the second bone fixation element 130 relative to the first slot 132 in the distal direction.

The second bone fixation element 130 can be a threaded fastener, rivet, nail, or adhesive. The second bone fixation element 130 can be similar to the first bone fixation element 124. The first bone fixation element 124 and the second bone fixation element 130 can be substantially identical (i.e., within manufacturing tolerance).

The first nail member 114 can further be configured to receive a third bone fixation element 131 (FIG. 5). The third bone fixation element 131 can be coupled to the second bone portion 120. For instance, the third bone fixation element 131 can be drilled through a hole in the second bone portion 120, or can form a hole through the second bone portion 120 as it is driven through the second bone portion 120. The third bone fixation element 131 can be translatably fixed to the second bone portion 120 with respect to relative movement along the longitudinal direction L The first nail member 114 can include a second slot 133 (FIG. 2) configured to receive the third bone fixation element 131. The first nail member 114 can include a second slot sidewall 135 can define the second slot 133. The second slot 133 can extend through the outer wall 122 of the first nail member 114. The second slot 133 can be elongate in the longitudinal direction L such that the first nail member 114 is moveable in the longitudinal direction L relative to the third bone fixation element 131 when the third bone fixation element 131 is within the second slot 133. In this regard, it should be appreciated that the first and second slots 132 and 133 can be elongate along respective directions of elongation that are parallel to each other. The second slot 133 can have a length L_{2B} in the longitudinal direction. The length L_{2B} can be greater than the length L₁. In some examples, the length L_{2B} is equal to the length L_{2A}. In other examples, one of the lengths L₂ₐ, L_{2B} are greater than the other of the lengths L₂ₐ, L_{2B}. The third bone fixation element 131 can be movable within the second slot 133 from a respective first position (FIG. 4) to a respective second position (FIG. 5). In one example, movement of the third bone fixation element 131 in the second slot 133 can be defined by a translation of the third bone fixation element 131 along the longitudinal direction L. Accordingly, the first nail member 114 can be movable relative to the third bone fixation element 131, and thus relative to the second bone portion 120, from the first position (FIG. 4) to the second position (FIG. 5). The third bone fixation element 131 can translate in the second slot 133 as the first nail member 114 moves relative to the third bone fixation element 131, and thus relative to the second bone portion 120. The third bone fixation element 131 can contact a distal portion of the second slot sidewall 135 in the first position. The third bone fixation element 131 can contact a proximal portion of the second slot sidewall 135 in the second position. Alternatively, the third bone fixation element 131 can be spaced from the proximal end of the second slot 133 in one or both of the first and second positions. The second position of the third bone fixation element 131 relative to the second slot 133 can be spaced from the first position of the third bone fixation element 131 relative to the second slot in the distal direction.

The third bone fixation element 131 can be a threaded fastener, rivet, nail, or adhesive. The third bone fixation element 131 can be similar to the first bone fixation element 124. The first bone fixation element 124 and the third bone fixation element 131 can be the same.

The outer wall 122 of the first nail member 114 can define a channel 136 configured to receive at least a portion of the second nail member 116. The second nail member 116 can be movable within the channel 136. In one example, the second nail member 116 can be movable within the channel 136 along the longitudinal direction. In particular, the outer wall 122 of the first nail member 114 can guide translation of the second nail member 116 in the channel 136. In this regard, the channel 136 can be elongate in the longitudinal direction L. The channel 136 can be elongate along the central axis A_{1.} The channel 136 can extend from the distal end 102 toward the proximal end 104. The channel 136 can extend from the distal end 102 to the proximal end 104. The channel 136 can extend through each of the distal end 102 and the proximal end 104.

The channel 136 can include a first channel portion 138 and a second channel portion 140. The first nail member 114 can include a seat 142 for a biasing element 164 (see FIG. 4). The seat 142 can separate the first channel portion 138 from the second channel portion 140. The first channel portion 138 can have a first channel width W₁. The width W₁ can be measured in a plane including the transverse direction T and the lateral direction A. The second channel portion 140 can have a second channel width W₂. The first channel width W₁ can be equal to the second channel width W₂. In other examples, the first channel width W₁ can be greater than the second channel width W₂. In still other examples, the first channel width W₁ can be less than the second channel width W₂. The first channel portion 138 can have a respective length L₃ in the longitudinal direction L. The second channel portion 140 can have a respective length L₄ in the longitudinal direction L. The length L₃ of the first channel portion 138 can be greater than the length L₄ of the second channel portion 140. The length L₃ of the first channel portion 138 can be a majority of the length of the first nail member 114. A ratio of the length L₃ of the first channel portion 138 to the length L₄ of the second channel portion 140 can be about 1:1, about 2:1, about 3:1, about 4:1, about 5:1, or about 6:1. The seat 142 and the outer wall 122 can define a single monolithic structure.

The first nail member 114 can define a passageway 144 that extends through the seat 142. For instance, the passageway 144 can extend through the seat 142 in the longitudinal direction L. The passageway 144 can be an opening that extends from the first channel portion 138 to the second channel portion 140 through the seat 142. The passageway 144 can have a width that is less than each of the first channel width W₁ and the second channel width W₂.

The second nail member 116 can be configured to be positioned in the channel 136. In some embodiments, the second nail member 116 can be positioned in the first channel portion 138. In other embodiments, the second nail member 116 can be positioned in the second channel portion 140. In other embodiments, the second nail member 116 can be positioned in both the first channel portion 138 and the second channel portion 140. Thus, the second nail member 116 can extend through the passageway 144 such that the second nail member 116 is positioned in each of the first channel portion 138 and the second channel portion 140.

The second nail member 116 can include a shaft 146 and a first end portion that extends from the shaft 146 in the proximal direction. The first end portion can be defined by a receiver 148. Thus, reference herein to the receiver 148 can apply with equal force and effect to the first end portion unless otherwise indicated. The shaft 146 can threadedly engage the receiver 148. The shaft 146 can be movable in the longitudinal direction L relative to the receiver 148 to couple the shaft 146 to the receive 148. The shaft 146 can be rotatable relative to the receiver 148 to couple the shaft 146 to the receiver 148. The shaft 146 can be rotatable about the central axis A₁ relative to the receiver 148. The shaft 146 can be fixed relative to the receiver 148 in the longitudinal direction L when the shaft 146 is coupled to the receiver 148. The first end portion can alternatively be monolithic with the shaft 146 or coupled to the shaft 146 in any manner as desired. The second nail member 116 can have a respective length L₅ in the longitudinal direction L. The length L₅ can be defined by the shaft 146 and the receiver 148. In some examples, the length L₅ of the second nail member 116 can be less than the length of the first nail member 114.

The shaft 146 can extend through the passageway 144 of the seat 142. The receiver 148 can be positioned in the second channel portion 140. The shaft 146 can extend from the second channel portion 140 through the passageway 144 and into the first channel portion 138. The shaft 146 can have a length L₆ that is less than the length L₃ of the first channel portion 138. A distal end 150 of the shaft 146 can be recessed with respect to the distal end 102 of the first nail member 114 in the proximal direction.

The shaft 146 can include a shaft body 137 and a second end portion that extends from the shaft body 137. The second end portion can be configured to receive the first bone fixation element 124. The second end portion can be defined by a fastening portion 152. Thus, reference herein to the fastening portion 152 can apply with equal force and effect to the second end portion, unless otherwise indicated. In some examples, the fastening portion 152 and the shaft body 137 can be monolithically formed. In other examples, the fastening portion 152 can be coupled to the shaft body 137 via threaded engagement, adhesive, weld, or fastener. The fastening portion 152 can have a width that is greater than the width of the shaft body. The fastening portion 152 and the receiver 148 can have equal widths. The fastening portion 152 can define a recess configured to receive at least a portion of the shaft 146.

The receiver 148 can have a width that is greater than the width of the passageway 144. Thus, the receiver 148 is unable to travel within the channel 136 through the passageway 144, and instead is configured to abut the seat 142. The receiver 148 can have a length L₇ that is less than the length L₄ of the second channel portion 140. The receiver 148 can be movable within the second channel portion 140. The receiver 148 can be movable in the longitudinal direction L within the second channel portion 140. In some embodiments, the receiver 148 can abut the seat 142 after the bone portions are reduced. In other embodiments, the receiver 148 is spaced from the seat 142 after the bone portions are reduced. The receiver 148 being spaced from the seat 142, for instance in the proximal direction, after the bone portions are reduced can allow the bone portions to apply a continuous compressive force against each other during the healing process. The receiver 148 can be movable within the second channel portion 140 while remaining within the second channel portion 140. In other embodiments, at least part of the receiver 148 can extend out of the second channel portion 140, for instance in the proximal direction. The length L₇ of the receiver 148 can be less than the length L₆ of the shaft 146. The receiver 148 and the fastening portion 152 can be disposed on opposite sides of a centerline the shaft 146 in the longitudinal direction L. The receiver 148 and the fastening portion 152 can be disposed on opposite ends of the shaft 146. The receiver 148 and the fastening portion 152 can be disposed on opposite ends of the shaft 146 in the longitudinal direction L.

The second nail member 116 can be fixed to the second bone portion 120 by the second bone fixation element 130 (see FIG. 5). The second bone fixation element 130 can fix the second nail member 116 relative to the second bone portion 120 in the longitudinal direction L. The second bone fixation element 130 can fix the second nail member 116 to the second bone portion 120 such that the second bone portion 120 moves relative to the first bone portion 118 in response to movement of the second nail member 116 relative to the first nail member 114.

The second nail member 116 can be configured to receive the second bone fixation element 130 (see FIG. 5). The second nail member 116 can include a second aperture 154 configured to receive the second bone fixation element 130. The second aperture 154 can define a second bone fixation hole. The second aperture 154 can extend through the second nail member 116 in a direction transverse to the longitudinal direction L. In some embodiments, the second aperture 154 extends through the second nail member 116. In other embodiments, the second aperture 154 extends from a first side of the second nail member 116 toward a second side of the second nail member 116 but does not extend completely through the second nail member 116. The second bone fixation element 130 can threadedly engage the second nail member 116 in the second aperture 154. In this regard, the second bone fixation element 130 and the second nail member 116 can be fixed to each other at least with respect to translation along the longitudinal direction L. The second bone fixation element 130 can be configured to fix the second nail member 116 to the second bone portion 120 in the longitudinal direction L when the second bone fixation element 130 is within the second aperture 154. Thus, the second bone fixation element 130 can fix the second bone portion 120 to the second nail member 116 at least with respect to translation along the longitudinal direction L. The first aperture 126 and second aperture 154 can have the same dimension in the longitudinal direction L.

The second aperture 154 can be aligned with at least a portion of the first slot 132 in the transverse direction T. In other examples, the second aperture 154 can be aligned with at least a portion of the first slot 132 in the lateral direction A or in any alternative direction perpendicular to the longitudinal direction L. The second aperture 154 can have a length in the longitudinal direction L that is less than the length L_{2A} of the first slot 132. The second aperture 154 can overlap the first slot 132. The second bone fixation element 130 can be configured to extend through each of the first slot 132 and the second aperture 154. The first nail member 114 can be movable in the longitudinal direction L relative to the second bone fixation element 130 while the second nail member 116 is fixed in the longitudinal direction L relative to the second bone fixation element 130.

The second nail member 116 can be further configured to receive the third bone fixation element 131 (see FIG. 5). The second nail member 116 can include a third aperture 155 configured to receive the third bone fixation element 131. The third aperture 155 can be a third bone fixation hole. The third aperture 155 can extend through the second nail member 116 in a direction transverse to the longitudinal direction L. In some embodiments, the third aperture 155 extends through the second nail member 116. In other embodiments, the third aperture 155 extends from a first side of the second nail member 116 toward a second side of the second nail member 116 but does not extend completely through the second nail member 116. The third bone fixation element 131 can threadedly engage the second nail member 116 in the third aperture 155. In this regard, the third bone fixation element 131 and the second nail member 116 can be fixed to each other at least with respect to translation along the longitudinal direction L. The third bone fixation element 131 can be configured to fix the second nail member 116 to the second bone portion 120 in the longitudinal direction L when the third bone fixation element 131 is within the third aperture 155. Thus, the third bone fixation element 131 can fix the second bone portion 120 and the second nail member 116 to each other at least with respect to translation along the longitudinal direction L. The second aperture 154 and third aperture 155 can have the same dimension in the longitudinal direction L.

The third aperture 155 can be aligned with at least a portion of the second slot 133 in the transverse direction T. In other examples, the third aperture 155 can be aligned with at least a portion of the second slot 132 in the lateral direction A or in any alternative direction perpendicular to the longitudinal direction L. The third aperture 155 can have a length in the longitudinal direction L that is less than the length L_{2B} of the second slot 133. The third aperture 155 can overlap the second slot 133. The third bone fixation element 131 can be configured to extend through each of the second slot 133 and the third aperture 155. The first nail member 114 can be movable in the longitudinal direction L relative to the third bone fixation element 131 while the second nail member 116 is fixed in the longitudinal direction L relative to the third bone fixation element 131.

The second nail member 116 can include a third slot 156 configured to receive the first bone fixation element 124 (see FIG. 4). The third slot 156 can extend through the second nail member 116. The third slot 156 can extend from a first side of the second nail member 116 toward a second side of the second nail member 116 without extending completely through the second nail member 116. Alternatively, the third slot 156 can extend completely through the second nail member 116. The third slot 156 can be elongate in the longitudinal direction L. The third slot 156 can be elongate in the longitudinal direction L such that the second nail member 116 is movable or translatable in the longitudinal direction L relative to the first bone fixation element 124 when the first bone fixation element 124 is extends through the third slot 156. The first bone fixation element 124 can be movable, such as translatable, along the longitudinal direction relative to the third slot 156 from a respective first position (FIG. 4) to a respective second position (FIG. 5). The first position of the first bone fixation element 124 can be offset in the distal direction relative to the third slot 156 with respect to the second position of the first bone fixation element 124. Thus, it should be appreciated that the second nail member 116 can be movable, such as translatable, along the longitudinal direction L relative to the first bone fixation element 124 and the first nail member 114 when the first bone fixation element 124 extends through the third slot 156. The second nail member 116 can be guided to translate along the longitudinal direction L relative to the first bone fixation element 124 and the first nail member 114 when the first bone fixation element 124 extends through the third slot 156. The first bone fixation element 124 can contact a distal end of the third slot 156 in the first position. The first bone fixation element 124 can contact a proximal end of the third slot 156 in the second position. Alternatively, the first bone fixation element 124 can be spaced from the proximal end of the third slot 156 in the second position. The first bone fixation element 124 can move relative to the third slot 156 as the first nail member 114 moves relative to the second nail member 116.

The second nail member 116 can be movable relative to the first nail member 114 from a respective first position (FIG. 5) to a respective second position (FIG. 4). A proximal end 162 of the second nail member 116 can be spaced from the proximal end 104 of the first nail member 114 by a first distance D₁ in the first position. The proximal end 162 can be spaced from the proximal end 104 by a second distance D₂ in the second position. The first distance D₁ can be greater than the second distance D₂. Otherwise stated, the second position of the second nail member 116 is offset from the first position of the second nail member 116 in the proximal direction.

The fixation device 100 can include a biasing element 164 configured to urge at least one of the first and second nail members 114, 116 to move, for instance to translate along the longitudinal direction L, relative to the other of the first and second nail members 114, 116. The biasing element 164 can be a spring. The biasing element 164 can be a resilient element (e.g., rubber). The biasing element 164 can be made from nickel-titanium (Nitinol) such that the biasing element 164 expands in response to temperature change. The biasing element 164 can be compressed gas. The biasing element 164 can be positioned in the channel 136. The biasing element 164 can be positioned in the first channel portion 138. The biasing element 164 can be compressible. The biasing element 164 can be compressed between the first nail member 114 and the second nail member 116. It should be appreciated that the fixation device 100 can alternatively be constructed such that the biasing element 164 is in tension between the first nail member 114 and the second nail member 116. The biasing element 164 can be positioned in the distal portion of the first nail member 114. The biasing element 164 can have a cylindrical cross-sectional shape. The biasing element 164 can define an internal opening such that the shaft 146 extends through the biasing element 164. The biasing element 164 can be a plurality of springs.

Referring to FIG. 3, the second nail member 116 can include a force receiving portion 158. The force receiving portion 158 can extend out from the shaft 146 along a direction that is perpendicular to the longitudinal direction L. The biasing element 164 can be captured between the force receiving portion 158 and the seat 142. When the second nail member 116 is in the second position, the biasing element 164 can exert a force against the force receiving portion 158 so as to cause relative movement between the first and second nail members 114, 116. In particular, the biasing element 164 can urge the second nail member 116 to move or translate with respect to the first nail member 114 in the distal direction. In some examples, the force receiving portion 158 is a flange that is fixed to the shaft 146. In other examples, the force receiving portion 158 is a collar that is coupled to the shaft 146. The force receiving portion 158 can be coupled to the shaft 146 by a fastener 160. The fastener 160 can extend through the force receiving portion 158 and into the shaft 146 such that the force receiving portion 158 is rotationally and axially fixed relative to the shaft 146. The fastener 160 can be a dowel, pin, screw, or rod. Alternatively, the force receiving portion 158 can be coupled to the shaft 146 by adhesive, weld, or press fit. The force receiving portion 158 can be detachably coupled to the shaft 146. The shaft 146 can include an opening configured to receive the fastener 160. The force receiving portion 158 can be positioned in the first channel portion 138. The force receiving portion 158 can have a width that is greater than the width of the shaft 146. The force receiving portion 158 can have a width that is greater than the width of the passageway 144.

The biasing element 164 can urge the second nail member 116 to move in the distal direction toward the distal end 102 (see FIG. 1) with respect to the first nail member 114. The biasing element 164 can cause relative movement between the first nail member 114 and the second nail member 116 so as to decrease a distance from the at least one first bone fixation hole, such as the first bone fixation hole 126 of the first nail member 114 (see FIG. 2) to the at least one second bone fixation hole, such as the second bone fixation hole 154 of the second nail member 116 along the longitudinal direction L or the central axis A₁. The biasing element 164 can move the first nail member 114 relative to the second nail member 116 such that the first bone fixation element 124 moves relative to the third slot 156. The biasing element 164 can move the first nail member 114 relative to the second nail member 116 such that the second bone fixation element 130 moves relative to the first slot 132.

The first bone portion 118 can be spaced from the second bone portion 120 in the longitudinal direction L prior to reducing the bone portions. The fixation device 100 can urge the bone portions 118, 120 to exert a force against each other even if the bone portions are not displaced from each other. The first bone portion 118 can move relative to the second bone portion 120 in response to urging from the biasing element 164. The biasing element 164 can move the first bone portion 118 into contact with the second bone portion 120. The biasing element 164 can move the first bone portion 118 into contact with the second bone portion 120 such that the first bone portion 118 and second bone portion 120 exert a compressive force against each other. The first bone portion 118 can move relative to the second bone portion 120 as the first bone fixation element 124 moves in the third slot 156 from the first position to the second position. The first bone portion 118 can move relative to the second bone portion 120 as the second bone fixation element 130 moves in the first slot 132 from the first position to the second position. The first bone fixation element 124 and second bone fixation element 130 can simultaneously move within the third slot 156 and the first slot 132, respectively.

A method of reducing the first and second bone portions 118, 120 can include the step of inserting the fixation device 100 into the medullary canal such that the first nail member 114 spans across an unreduced bone gap, such as a fracture, between the first bone portion 118 and the second bone portion 120. The first bone fixation element 124 can be inserted into the first aperture 126 of the first nail member 114, and the third slot 156 of the second nail member 116. The first bone fixation element 124 can also be inserted into the first bone hole 128. The first nail member 114 can be fixed to the first bone portion 118 with respect to longitudinal movement when the first bone fixation element 124 is extends through the first aperture 126. The first bone fixation element 124 can contact a proximal end of the third slot 156 when the fixation device is in an initial configuration shown in FIG. 3. The receiver 148 can be in contact with the seat 142 in the initial configuration (FIG. 3).

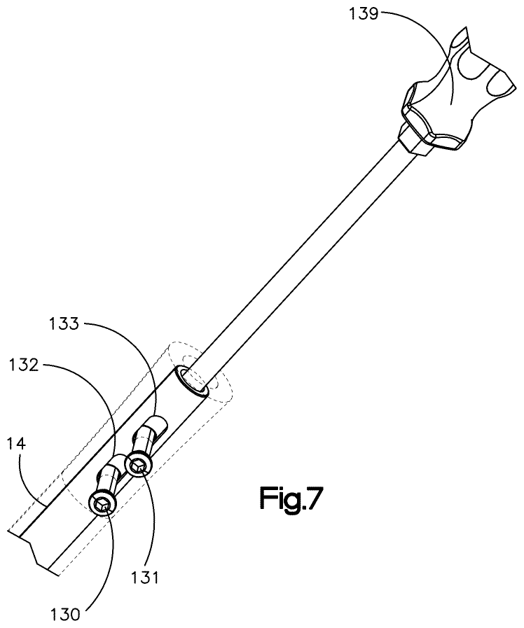

The second nail member 116 can then be moved relative to the first nail member 114 from the initial configuration to an intermediate configuration shown in FIG. 4. The second nail member 116 can move proximally in the longitudinal direction L from the initial configuration to the intermediate configuration. In some examples, the second nail member 116 can be moved from the initial configuration to the intermediate configuration by employing a spring compression pull tool 139 (see FIG. 7). The pull tool 139 can be threaded into the receiver 114 to pull the receiver 148, and thus the force receiving portion 158, proximally, thereby compressing the biasing element 164 and placing the device 100 in the intermediate configuration shown at FIG. 4. The receiver 148 can be spaced from the seat 142 in the intermediate configuration. The first bone fixation element 124 can contact a distal end of the third slot 156 when the fixation device 100 is in the intermediate configuration. Movement of the second nail member 116 proximally relative to the first nail member 114 can increase the potential energy of the biasing element 164. The biasing element 164 can be compressed between the force receiving portion 158 and the seat 142 as the second nail member 116 moves relative to the first nail member 114 from the initial configuration to the intermediate configuration.

The second bone fixation element 130 can be inserted into the first slot 132 and the second aperture 154 when the fixation device 100 is in the intermediate configuration. The second bone fixation element 130 can be inserted into the first slot 132, the second aperture 154, and a second bone hole 166 in the second bone portion 120. The second bone fixation element 130 can contact a proximal end of the first slot 132 when the fixation device 100 is in the intermediate configuration. The third bone fixation element 131 can be inserted into the second slot 133 and the third aperture 155 when the fixation device 100 is in the intermediate configuration. The third bone fixation element 131 can be inserted into the second slot 133, the second aperture 155, and a third bone hole 167 in the second bone portion 120. The third bone fixation element 131 can contact a proximal end of the second slot 133 when the fixation device 100 is in the intermediate configuration. The second nail member 116 can be fixed in the longitudinal direction relative to the first nail member 114 when the fixation device 100 is in the intermediate configuration. The second nail member 116 can be fixed relative to the first nail member 114 by a restraint.

The restraint can be deactivated such that the fixation device 100 transitions to a reduced configuration shown at FIG. 5. The second nail member 116 can move relative to the first nail member 118 as the fixation device 100 transitions from the intermediate configuration to the reduced configuration. The biasing element 164 can move the second nail member 116 relative to the first nail member 114 once the restraint is deactivated. The first bone fixation element 124 can be spaced from the proximal end of the third slot 156 when the fixation device 100 is in the reduced configuration. The second bone fixation element 130 can be spaced from the distal end of the first slot 132 when the fixation device 100 is in the reduced configuration. The first bone portion 118 can move relative to the second bone portion 120 in response to movement of the first nail member 114 relative to the second nail member 116. The first bone portion 118 and second bone portion 120 can exert a force against each other when the fixation device 100 is in the reduced configuration. The distal end of the second bone portion 120 can exert a compressive force against the proximal end of the first bone portion 118 when the fixation device 100 is in the reduced configuration. The first bone fixation element 124 and second bone fixation element 130 being spaced from the ends of the first slot 132 and the third slot 156, respectively, can allow the first bone portion 118 and second bone portion 120 to exert a continuous compressive force against each other. The receiver 148 can be spaced from the seat 142 when the fixation device 100 is in the reduced configuration. Alternatively, the receiver 148 can contact the seat 142 when the fixation device 100 is in the reduced configuration.

A method of assembling the fixation device 100 can include a step 190 of providing the first nail member 114 and the second nail member 116. The method can include a step 192 of coupling the second nail member 116 to the first nail member 114. The coupling step 192 can include coupling the fastening portion 152 to the shaft 146. Coupling the fastening portion 152 to the shaft can include rotating one of the fastening portion 152 and the shaft 146 relative to the other of the fastening portion 152 and the shaft 146. The coupling step 192 can include coupling the force receiving portion 158 to the shaft 146. Coupling the force receiving portion 158 to the shaft can include inserting a collar fastener 160 into an opening in the force receiving portion 158 to fix the force receiving portion 158 to the shaft 146. The coupling step 192 can include coupling the biasing element 164 to the second nail member 116. Coupling the biasing element 164 to the second nail member 116 can include inserting the shaft 146 into an opening in the biasing element 164. Alternatively, the coupling step 192 can include positioning the biasing element 164 in the channel 136 prior to coupling the biasing element 164 to the second nail member 116. The coupling step 192 can include positioning the second nail member 116 in the channel 136 of the first nail member. Positioning the second nail member 116 in the recess can include moving at least a portion of the shaft 146 through the passageway 144 in the seat 142. Positioning the second nail member 116 in the recess can include coupling the receiver 148 to the shaft 146. Coupling the receiver 148 to the shaft 146 can include rotating one of the receiver and the shaft 146 relative to the other of the receiver 148 and the shaft 146.

The method can include a step 194 of moving the second nail member 116 relative to the first nail member 114. The moving step 194 can include moving the second nail member 116 in the longitudinal direction L relative to the first nail member 114. The moving step 194 can include increasing the potential energy of the biasing element 164.

While systems and methods have been described in connection with the various embodiments of the various figures, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

When values are expressed as approximations by use of the antecedent “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function, and the person skilled in the art will be able to interpret it as such. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, reference to values stated in ranges includes each and every value within that range.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention. Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

What is claimed:

1. An intramedullary nail that extends along a central axis, the intramedullary nail comprising:
a first nail member configured to be positioned in a medullary canal of a bone, wherein the first nail member defines at least one first bone fixation hole configured to receive a respective at least one first bone fixation element so as to couple the first nail member to a first bone portion;
a second nail member configured to be at least partially inserted into the first nail member, wherein the second nail member defines at least one second bone fixation hole configured to receive a respective at least one second bone fixation element so as to couple the second nail member to a second bone portion that is spaced from the first bone portion;
a biasing element configured to apply a biasing force to at least one of the first and second nail members so as to apply compression between the first and second bone portions when the first nail member is coupled to the first bone portion and the second nail member is coupled to the second bone portion.

2. The intramedullary nail of claim 1, wherein the first nail member includes a channel configured to receive at least a portion of the second nail member.

3. The intramedullary nail of claim 2, wherein at least a portion of the second nail member is disposed in the channel of the first nail member.

4. The intramedullary nail of claim 2, wherein the second nail member is disposed in the channel of the first nail member.

5. The intramedullary nail of claim 1, wherein a first end of the biasing element is supported by a seat within the first nail member, a second end of the biasing element bears against the second nail member in a select direction, and the biasing force is applied to the second nail member in the select direction.

6. The intramedullary nail of claim 5, wherein a passageway extends through the seat in the longitudinal direction.

7. The intramedullary nail of claim 6, wherein at least a portion of the second nail member is within the passageway.

8. The intramedullary nail of claim 5, wherein the biasing element is compressed between the seat and the second nail member.

9. The intramedullary nail of claim 1, wherein the second nail member includes a shaft and a receiver configured to couple to the shaft, the shaft having a shaft width in the lateral direction and the receiver having a receiver width that is greater than the shaft width.

10. The intramedullary nail of claim 9, wherein the shaft extends through the channel.

11. The intramedullary nail of claim 9, further comprising a collar coupled to the shaft, the collar having a collar width that is greater than the shaft width.

12. The intramedullary nail of claim 11, wherein the biasing element is positioned between the seat and the collar.

13. The intramedullary nail of claim 11, wherein the collar is fixed to the shaft.

14. The intramedullary nail of claim 1, wherein the first nail member includes a first slot that is elongate along a longitudinal axis of the first nail member and the second bone fixation element is positioned within the first slot such that the first nail member is movable relative to the second bone fixation element.

15. The intramedullary nail of claim 14, wherein the second nail member includes a second slot that is elongate along a longitudinal axis of the second nail member and the first bone fixation element is positioned within the second slot such that the second nail member is movable relative to the first bone fixation element.

16. The intramedullary nail of claim 1, wherein the intramedullary nail is transitionable from a first configuration to a second configuration,
wherein in the first configuration a distal end of the first nail member is spaced from a distal end of the second nail member by an initial distance, and
wherein in the second configuration the distal end of the first nail member is spaced from the distal end of the second nail member by a compressed distance that is greater than the initial distance.

17. The intramedullary nail of claim 1, wherein a length of the intramedullary nail is the same in the first configuration and the second configuration, wherein the length is measured along the central axis from a first terminal end of the first nail member to a second terminal end of the second nail member opposite the first terminal end.

18. The intramedullary nail of claim 1, wherein the first nail member has a fixed length in the longitudinal direction.

19. A method of coupling an intramedullary nail that extends along a central axis to a first bone portion and a second bone portion, the intramedullary nail including a first nail member and a second nail member, the method comprising:
positioning the first nail member in a medullary canal of at least one bone that defines the first and second bone portions, wherein the first nail member defines at least one first bone fixation hole;
inserting a respective at least one first bone fixation element into the at least one first bone fixation hole so as to couple the first nail member to the first bone portion,
inserting a respective at least one second bone fixation element into at least one second bone fixation hole in the second nail member so as to couple the second nail member to the second bone portion; and
applying a biasing force to at least one of the first and second nail members so as to apply compression between the first and second bone portions.

20. The method of claim 19, wherein applying step is applied by a biasing element that is supported by a seat within the first nail member and bears against the second nail member in a select direction, and the biasing force is applied to the second nail member in the select direction.
